(19) United States
(12) Patent Application Publication
(10) Pub. No.: US 2025/0059195 A1
(43) Pub. Date: Feb. 20, 2025

(54) TRIAZOLONE DERIVATIVE SALT AS NEUTROPHIL ELASTASE INHIBITOR

(71) Applicant: CHIESI FARMACEUTICI S.P.A., Parma (IT)

(72) Inventors: Irene BASSANETTI, Parma (IT); Francesco AMADEI, Parma (IT)

(73) Assignee: CHIESI FARMACEUTICI S.P.A., Parma (IT)

(21) Appl. No.: 18/720,015

(22) PCT Filed: Dec. 21, 2022
(86) PCT No.: PCT/EP2022/087155

§ 371 Date: Jun. 14, 2024

(30) Foreign Application Priority Data
Dec. 22, 2021 (EP) 21217146.6

(51) Int. Cl. C07D 487/04; A61K 31/519; A61P 11/00; C07C 65/11

(52) U.S. Cl. CPC C07D 487/04; A61K 31/519; A61P 11/00; C07C 65/11; C07C 2602/10

(57) ABSTRACT

The present invention generally relates to a novel triazolone derivative salt particularly useful as neutrophil elastase inhibitor and to its use as medicament; the invention also relates to its synthesis process and pharmaceutical compositions thereof. The invention also relates to the process for the isolation by crystallization of the compound (I). The invention also relates to a crystal form of a compound of formula (I).

### FIELD OF INVENTION

The present invention generally relates to a novel triazolone derivative salt particularly useful as neutrophil elastase inhibitor and to its use as medicament; the invention also relates to its synthesis process and pharmaceutical compositions thereof. The invention also relates to the process for the isolation by crystallization of the compound (I). The invention also relates to a crystal form of a compound of formula (I).

### BACKGROUND OF THE INVENTION

Human neutrophil elastase (HNE) is a 32 kDa serine proteinase found in the azurophilic granules of neutrophils. It has a role in the degradation of a wide range of extracellular matrix proteins, including fibronectin, laminin, proteoglycans, Type III and Type IV collagens as well as elastin (Bieth, G. In Regulation of Matrix accumulation, Mecham, R. P. (Eds), Academic Press, NY, USA 1986, 217-306). HNE has long been considered to play an important role in homeostasis through repair and disposal of damaged tissues via degradation of the tissue structural proteins. It is also relevant in the defense against bacterial invasion by means of degradation of the bacterial body. In addition to its effects on matrix tissues, HNE has been implicated in the upregulation of IL-8 gene expression and also induces IL-8 release from the epithelial cells of the lung. In animal models of Chronic Obstructive Pulmonary Disease induced by tobacco smoke exposure both small molecule inhibitors and protein inhibitors of HNE inhibit the inflammatory response and the development of emphysema (Wright, J. L. et al. Am. J. Respir. Crit. Care Med. 2002, 166, 954-960; Churg, A. et al. Am. J. Respir. Crit. Care Med. 2003, 168, 199-207). Thus, HNE it is a key mediator of lung tissue degradation and inflammation (K. M. Heutinck, I. J. ten Berge, C. E. Hack, J. Hamann, A. T. Rowshani, Mol. Immunol., 47 (11-12) (2010), 1943-55), and it may play a role both in matrix destruction and in amplifying inflammatory responses in chronic respiratory diseases where neutrophil influx is a characteristic feature. Indeed, HNE is believed to play a role in several pulmonary diseases, including chronic obstructive pulmonary disease (COPD), cystic fibrosis (CF), acute respiratory distress syndrome (ARDS), pulmonary emphysema, pneumonia, pulmonary fibrosis. It is also implicated in several cardiovascular diseases in which tissue remodelling is involved, for example, in heart failure and the generation of ischaemic tissue injury following acute myocardial infarction.

Excess HNE activity has been implicated in the pathology of inflammatory pulmonary diseases, including bronchiectasis (BE), so identifying this protein as a target for drug development (B. Schaaf, A. Wieghorst, S. P. Aries, K. Dalhoff, J. Braun, Respiration, 67 (1) (2000), 52-59).

Several human neutrophil inhibitors have been disclosed so far in the art.

In particular, International Patent Applications WO2011/110858 and WO2011/110859 describe some pyrimidine derivatives having human neutrophil elastase inhibitory properties and their use in therapy.

WO 2014/095700 describes triazolone derivatives having human neutrophil elastase inhibitory properties and their use in therapy and, in particular, some salts of the compound (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium as potent neutrophil elastase inhibitors.

Although several HNE inhibitors have been disclosed so far as above reported, there is still a need for further HNE inhibitors.

Particularly advantageous would be the identification of further HNE inhibitors endowed with a safer profile, suitable for administration, in particular as an inhalation treatment, also in terms of patients' tolerability and local adverse effects profile.

The present invention addresses the above mentioned needs by providing the compound of the invention, (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium xinafoate of formula (I).

### SUMMARY OF THE INVENTION

In a first aspect, the present invention provides (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium xinafoate of formula (I)

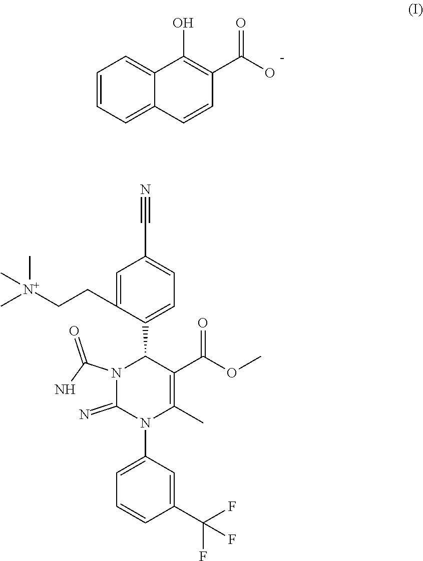

In a second aspect, the invention provides a pharmaceutical composition comprising (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]phenyl}-ethyl)-trimethyl-ammonium xinafoate of formula (I) and one or more pharmaceutically acceptable carriers and/or excipients.

In a further aspect, the invention provides (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium xinafoate of formula (I) for use as a medicament.

In a still further aspect, the invention provides a pharmaceutical composition comprising (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium xinafoate of formula (I) and one or more pharmaceutically acceptable carriers and/or excipients for use as a medicament.

In another aspect, the invention provides the use of (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium xinafoate of formula (I) for the manufacture of a medicament.

In another aspect, the invention provides (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium xinafoate of formula (I) for use in the prevention and/or treatment of an inflammatory or obstructive respiratory disease.

In another aspect, the invention provides (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium xinafoate of formula (I) for the manufacture of a medicament for the prevention and/or treatment of an inflammatory or obstructive respiratory disease.

In a further aspect, the invention provides a pharmaceutical composition comprising (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium xinafoate of formula (I) and one or more pharmaceutically acceptable carriers and/or excipients for use in the prevention and/or treatment of an inflammatory or obstructive respiratory disease.

In a further aspect, the invention provides the use of a pharmaceutical composition comprising (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium xinafoate of formula (I) and one or more pharmaceutically acceptable carriers and/or excipients in the manufacture of a medicament for the prevention and/or treatment of an inflammatory or obstructive respiratory disease.

In another aspect, the present invention provides a method for preventing and/or treating an inflammatory or obstructive respiratory disease, the method comprising administering an effective amount of (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium xinafoate of formula (I).

In a further aspect, the present invention provides a method for preventing and/or treating an inflammatory or obstructive respiratory disease, the method comprising administering an effective amount of pharmaceutical composition comprising (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium xinafoate of formula (I) and one or more pharmaceutically acceptable carriers and/or excipients.

In another aspect, the present invention provides a process for the preparation of the compound of formula (I), by reacting the triazolone derivative of formula (II) with a xinafoate salt of formula (III)

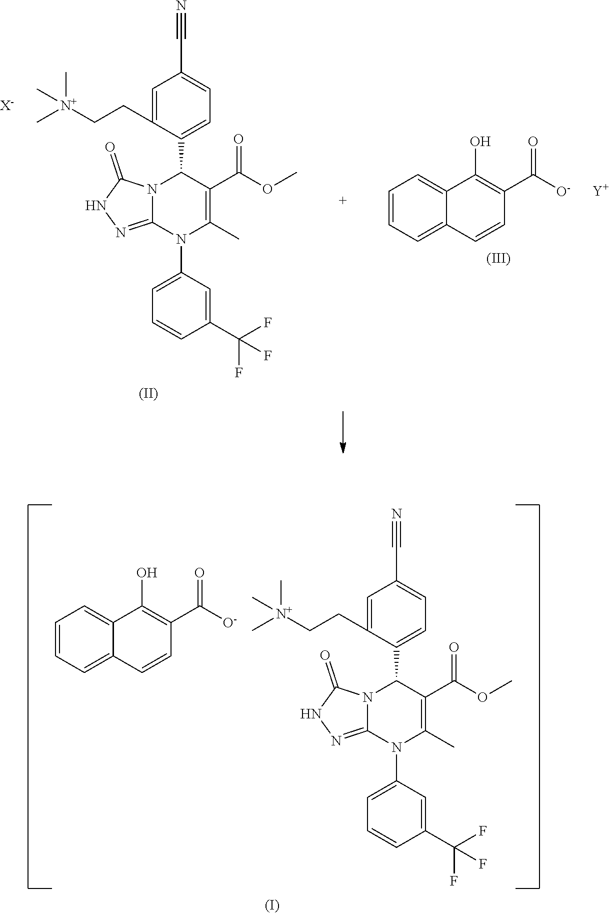

said process comprising the steps of:
   1) dissolving in water (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium salt (II)

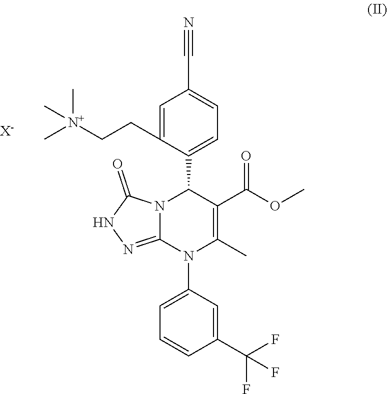

wherein X⁻ is any organic or inorganic anion,
   2) adding a solution of a xinafoate salt of formula (III)

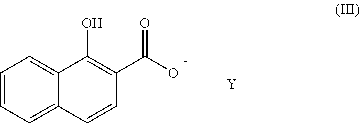

wherein Y⁺ is an alkaline or alkaline earth metal cation.

In a further aspect, the present invention also refers to a process for the preparation of the compound of formula (I), which further comprises step 3) of washing the compound of formula (I) obtained according to steps 1) and 2) with one or more aqueous or organic solvents.

In another aspect, the present invention refers to a crystal form of a compound of formula (I), wherein said crystal is characterized by at least one of the following XRPD peaks: 8.6, 9.9, 23.2±0.2 degrees/2 theta [Cu Kα radiation (λ=1.5406 Å)].

In another aspect, the present invention refers to a crystal form of a compound of formula (I) wherein said crystal is obtained according to steps 1) to 3) as above defined.

In a further aspect, the invention provides a crystal form of a compound of formula (I) for use as a medicament.

In a still further aspect, the invention provides a pharmaceutical composition comprising a crystal form of a compound of formula (I) and one or more pharmaceutically acceptable carriers and/or excipients for use as a medicament.

In another aspect, the invention provides the use of a crystal form of a compound of formula (I) as defined above for the manufacture of a medicament for the prevention and/or treatment of an inflammatory or obstructive respiratory disease.

In a further aspect, the invention provides a pharmaceutical composition comprising a crystal form of a compound of formula (I) as defined above and one or more pharmaceutically acceptable carriers and/or excipients for use in the prevention and/or treatment of an inflammatory or obstructive respiratory disease.

In a further aspect, the invention provides the use of a pharmaceutical composition comprising a crystal form of a compound of formula (I) as defined above and one or more pharmaceutically acceptable carriers and/or excipients for the manufacture of a medicament for the prevention and/or treatment of an inflammatory or obstructive respiratory disease.

In another aspect, the present invention provides a method for preventing and/or treating an inflammatory or obstructive respiratory disease, the method comprising administering an effective amount of to a crystal form of a compound of formula (I) as defined above.

In a further aspect, the present invention provides a method for preventing and/or treating an inflammatory or obstructive respiratory disease, the method comprising administering an effective amount of pharmaceutical composition comprising a crystal form of a compound of formula (I) as defined above and one or more pharmaceutically acceptable carriers and/or excipients.

### BRIEF DESCRIPTION OF THE FIGURES

FIG. 1: XRPD of compound (I). 2 Theta (°) corresponds to 2 Theta (degrees).

FIG. 2: ¹H NMR of compound (I)

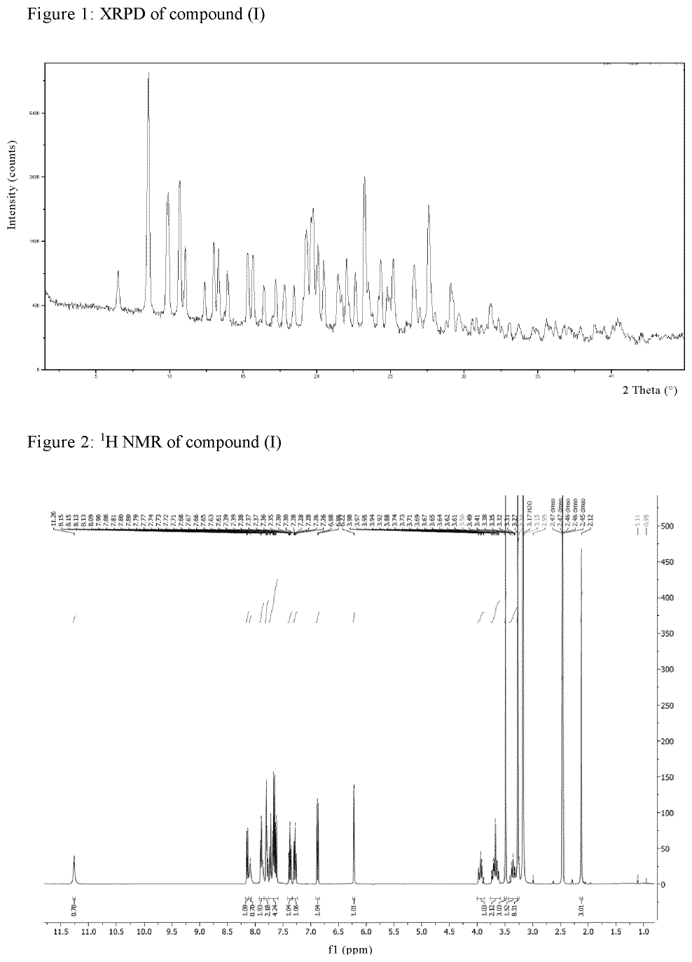

### DETAILED DESCRIPTION OF THE INVENTION

### Definitions

Unless defined otherwise, all technical and scientific terms used herein have the same meanings as commonly understood by the skilled in the art.

The term “compound of the invention” refers to (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium xinafoate of formula (I).

The term “xinafoate”, also called hydroxy-naphtoate or salt of 1-hydroxy-2-naphthalene, refers to the salt from hydroxynaphtoate anion, where the stoichiometric ratio between 2-{5-Cyano-2-[(R)-6-methoxycarbonyl-7-methyl-3-oxo-8-(3-trifluoromethyl-phenyl)-2,3,5,8-tetrahydro-[1,2,4]triazole [4,3-a]pyrimidine-5-yl]-phenyl}-ethyl)-trimethyl-ammonium cation and hydroxynaphtoate anion is 1:1.

The terms “alkaline or alkaline earth metal cation” are intended to indicate cations of metal elements selected from sodium, potassium, magnesium and calcium.

The compound of the invention has one stereogenic center, namely represented by the carbon atom (1) with an asterisk * below and therefore can exist as optical stereoisomers.

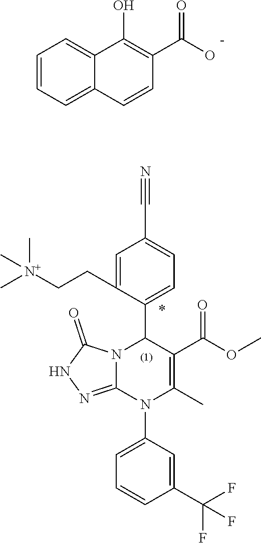

It is to be understood that besides the compound of the invention of formula (I) showing a preferred (R) configuration on carbon atom (1), the racemic form and enantiomer (S) are encompassed within the scope of the present invention.

The term “composition”, as in pharmaceutical composition, is intended to encompass a product comprising the active ingredient and any pharmaceutically acceptable excipient or carrier, as well as any product which results, directly or indirectly, from combination, complexation or aggregation of any two or more of the ingredients, or from dissociation of one or more of the ingredients, or from other types of reactions or interactions of one or more of the ingredients.

Accordingly, the pharmaceutical compositions of the invention comprehend any type of composition made by admixing the compound of the invention and pharmaceutically acceptable excipients and/or carriers.

The xinafoate salt as indicated in formula (I), is characterized by having physico-chemical features particularly suitable for administration, also in terms of patients' tolerability and local adverse effects profile.

Advantageously, in this respect, the xinafoate salt according to compound of formula (I) of the invention shows:
1) an unexpected, superior activity in a pharmacodynamic model, with respect to the methanesulfonate salt, when administered as dry powder, as described in Example 2.1 of the Biological assay. This is particularly unexpected in view of the lower solubility of the xinafoate salt with respect to methanesulfonate salt, and
2) a very good irritative profile in the HOP (Head Out Plethysmography) test in conscious rats, in comparison with methanesulfonate, bromide and acetate salts, when administered as dry powder. This suggests a possible safer profile also in humans, as described and commented in the present Example 2.2.

In consideration of the above, the xinafoate salt of (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium maintains, despite its lower solubility with respect to the methanesulfonate salt, a good efficacy in inhibiting HNE in-vivo and maintains a good efficacy also when administered as dry powder formulation.

Moreover, the Head-out plethysmography (HOP) assay showed that conversely to the other salts methanesulfonate, bromide and acetate of (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium, the xinafoate salt of formula (I), induces improved lung function parameters. This suggests that this salt is particularly suitable for administration, also in terms of patients' tolerability and local adverse effects profile.

In consideration of the above, the xinafoate salt of (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium maintains a good efficacy in inhibiting HNE in-vivo also and in particular when administered as dry powder formulation.

As mentioned above, the present invention provides (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium xinafoate of formula (I)

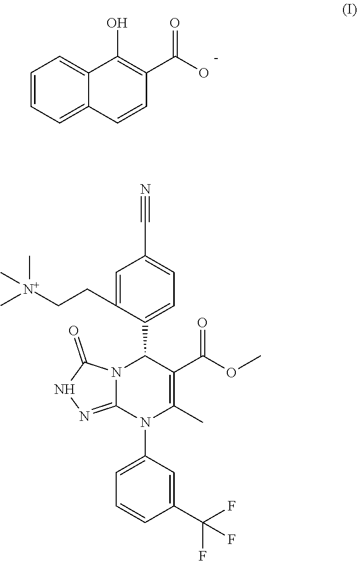

The present invention also refers to a process for the preparation of the compound of formula (I), by reacting the salt of formula (II) with a xinafoate salt of formula (III) as above identified, said process comprising the steps of:
1) dissolving in water (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium salt (II) wherein X⁻ is an organic or inorganic anion,
2) adding a solution of a xinafoate salt of formula (III), wherein Y⁺ is an alkaline or alkaline earth metal cation.

The present invention also refers to a process for the preparation of the compound of formula (I) by reacting a compound of formula (II), wherein X⁻ is an organic or inorganic anion, preferably selected from the group consisting of methanesulfonate, acetate, iodide and bromide.

In another preferred embodiment, the present invention provides a process for the preparation of a compound of formula (I) by reacting a compound of formula (II), wherein X⁻ is bromide.

In another preferred embodiment, the present invention provides a process for the preparation of a compound of formula (I) by reacting a compound of formula (II), wherein X⁻ is acetate.

In another preferred embodiment, the present invention provides a process for the preparation of the compound of formula (I), by reacting a compound of formula (II), wherein X⁻ is methanesulfonate.

In a further preferred embodiment, the present invention provides a process for the preparation of the compound of formula (I), by reacting a xinafoate salt of formula (III), wherein Y⁺ is sodium or potassium, being sodium even more preferred.

In a still further preferred embodiment, the present invention also provides a process for the preparation of the compound of formula (I), by reacting a compound of formula (II) with a xinafoate salt of formula (III), wherein the molar ration between (II) and (III) is 1:1.

In another preferred embodiment, the present invention provides a process for the preparation of the compound of formula (I), which further comprises step 3) of washing the compound of formula (I) obtained according to steps 1) and 2) with one or more aqueous or organic solvents. The solvents are preferably water, acetone, or a mixture thereof.

In a further preferred embodiment, the present invention provides a process for the preparation of the compound of formula (I) wherein the solvents used for washing the compound of formula (I) obtained according to steps 1) and 2) are aqueous or organic solvents. Preferably the solvents are water, acetone or a mixture thereof.

In another preferred embodiment, the present invention provides a crystal form of a compound of formula (I), wherein said crystal form is characterized by at least one of the following XRPD peaks: 8.6, 9.9 and 23.2±0.2 degrees/2 theta [Cu Kα radiation (λ=1.5406 Å)].

In another embodiment, the present invention provides a crystal form of a compound of formula (I), wherein said crystal form is characterized by the following XRPD peaks: 8.6, 9.9 and 23.2±0.2 degrees/2 theta [Cu Kα radiation (λ=1.5406 Å)].

In another preferred embodiment, the present invention provides a crystal form of a compound of formula (I), wherein said crystal form is characterized by the following XRPD peaks: 8.6, 9.9, 10.7, 11.0 and 23.2±0.2 degrees/2 theta [Cu Kα radiation (λ=1.5406 Å)].

In another preferred embodiment, the present invention provides a crystal form of a compound of formula (I), wherein said crystal form is characterized by the following XRPD peaks: 8.6, 9.9, 10.7, 11.0, 13.0, 15.3, 19.3, 19.7, 23.2 and 27.6±0.2 degrees/2 theta [Cu Kα radiation (λ=1.5406 Å)].

In another preferred embodiment, the present invention provides a crystal form of a compound of formula (I) preferably said crystal is obtained according to steps 1) to 3) as defined above.

In another preferred embodiment, the present invention provides the crystal form, for use in the prevention and/or treatment of an inflammatory or obstructive respiratory disease.

In a further preferred embodiment, the present invention provides the crystal form as defined above, for use in the prevention and/or treatment of wherein the inflammatory or obstructive respiratory diseases are selected from: asthma, chronic obstructive pulmonary disease (COPD), bronchiectasis, chronic bronchitis, lung fibrosis, idiopathic pulmonary fibrosis, pneumonia, acute respiratory distress syndrome (ARDS), pulmonary emphysema, smoking-induced emphysema and cystic fibrosis.

In an even further preferred embodiment, the present invention provides a pharmaceutical composition for inhalation comprising the crystal form of a compound of formula (I), in combination with suitable carriers and/or excipients.

The present invention is also directed to a pharmaceutical composition comprising the compound of formula (I) and one or more pharmaceutically acceptable carriers and/or excipients.

Suitable excipients can be selected among those in the art, and they can include carriers, diluents, wetting agents, emulsifying agents, binders, coatings, fillers, glidants, lubricants, disintegrants, preservatives, surfactants, pH buffering substances and the like. Examples of excipients and their use are provided in the Handbook of Pharmaceutical Excipients, 5th ed. (2006), Ed. Rowe et al., Pharmaceutical Press.

The most suitable dosage level may be determined by any known suitable method. It will be understood, however, that the specific amount for any particular patient will depend upon a variety of factors, including the activity of the compound of formula (I), the age, body weight, diet, general health and sex of the patient, time of administration, the route of administration, the rate of excretion, the use of any other drugs, and the severity of the disease to be treated.

For delivery by inhalation, the active compound of formula (I) is preferably in the form of microparticles. They may be prepared by a variety of techniques, including spray-drying, freeze-drying and micronization.

In one embodiment, a composition of the invention is prepared as a suspension, suitable for delivery from a nebulizer or as an aerosol in a liquid propellant, even more preferably for use in a pressurized metered dose inhaler (pMDI). Suitable propellants for use in a pMDI are known to the skilled person, and include HFA-227, preferably HFA-134a and more preferably HFA152a.

In a preferred embodiment, a composition of the invention is in dry powder form, for delivery using a dry powder inhaler (DPI).

Microparticles for delivery by administration may be formulated with excipients that aid delivery and release. For example, in a dry powder formulation, microparticles may be formulated with large carrier particles that aid flow from the DPI into the lung. Suitable carrier particles are known in the art and include e.g. lactose particles.

The agents of the invention may be administered in inhaled form. Aerosol generation can be carried out using, for example, pressure-driven jet atomizers or ultrasonic atomizers, preferably using propellant-driven metered aerosols or propellant-free administration of micronized active compound of formula (I) from, for example, inhalation capsules or other “dry powder” delivery systems.

As above described, the present invention is directed to the compound of general formula (I) for use as a medicament.

According to a preferred embodiment, the present invention refers to the use of the xinafoate salt of formula (I) for the preparation of a medicament for the treatment of an inflammatory or obstructive pulmonary disease, preferably the disease is selected from: asthma, chronic obstructive pulmonary disease (COPD), bronchiectasis, chronic bronchitis, lung fibrosis, idiopathic pulmonary fibrosis, pneumonia, acute respiratory distress syndrome (ARDS), pulmonary emphysema, smoking-induced emphysema and cystic fibrosis.

The present invention is also directed to a pharmaceutical composition comprising the compound of formula (I) and one or more pharmaceutically acceptable carriers and/or excipients for use as a medicament.

In a preferred embodiment, the present invention is directed to the compound of formula (I) for use for the prevention and/or treatment of an inflammatory or obstructive respiratory disease.

In another preferred embodiment, the present invention is directed to a pharmaceutical composition comprising the compound of formula (I) and one or more pharmaceutically acceptable carriers and/or excipients, for use for the prevention and/or treatment of an inflammatory or obstructive respiratory disease.

In a further preferred embodiment, the present invention provides a method for preventing and/or treating an inflammatory or obstructive respiratory disease, the method comprising administering an effective amount of (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium xinafoate of formula (I).

In another preferred embodiment, the present invention provides a method for preventing and/or treating an inflammatory or obstructive respiratory disease, the method comprising administering an effective amount of pharmaceutical composition comprising (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium xinafoate of formula (I) and one or more pharmaceutically acceptable carriers and/or excipients.

In a still further preferred embodiment, the inflammatory or obstructive respiratory diseases mentioned above are selected from asthma, chronic obstructive pulmonary disease (COPD), bronchiectasis, chronic bronchitis, lung fibrosis, idiopathic pulmonary fibrosis, pneumonia, acute respiratory distress syndrome (ARDS), pulmonary emphysema, smoking-induced emphysema and cystic fibrosis.

Any suitable route of administration may be employed for providing a mammal, especially a human, with an effective dosage of the compound of formula (I).

The magnitude of prophylactic or therapeutic dose of the compound of formula (I) will, of course, vary with the nature of the severity of the condition to be treated and with its route of administration, and will generally be determined by clinical trial as required in the pharmaceutical art.

It will also vary according to the age, weight and response of the individual patient.

In therapeutic use, the compound of formula (I) may be administered by any convenient, suitable or effective route.

Suitable routes of administration are known, and include oral, intravenous, rectal, parenteral, topical, ocular, nasal, buccal and pulmonary (by inhalation).

The active compound of formula (I) may be dosed as described depending on the inhaler system used. In addition to the active compound, the administration forms may additionally contain excipients, such as, for example, propellants (e.g. Frigen in the case of metered aerosols), surface-active substances, emulsifiers, stabilizers, preservatives, flavorings, fillers (e.g. lactose in the case of powder inhalers) or, if appropriate, further active compounds.

For the purposes of inhalation, a large number of systems are available with which aerosols of optimum particle size can be generated and administered, using an inhalation technique which is appropriate for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhalers for example as described EP-A-0505321).

In a more preferred embodiment, the invention provides a process for the preparation of the compound of the invention of formula (I), according to general synthetic route reported in Scheme A here below

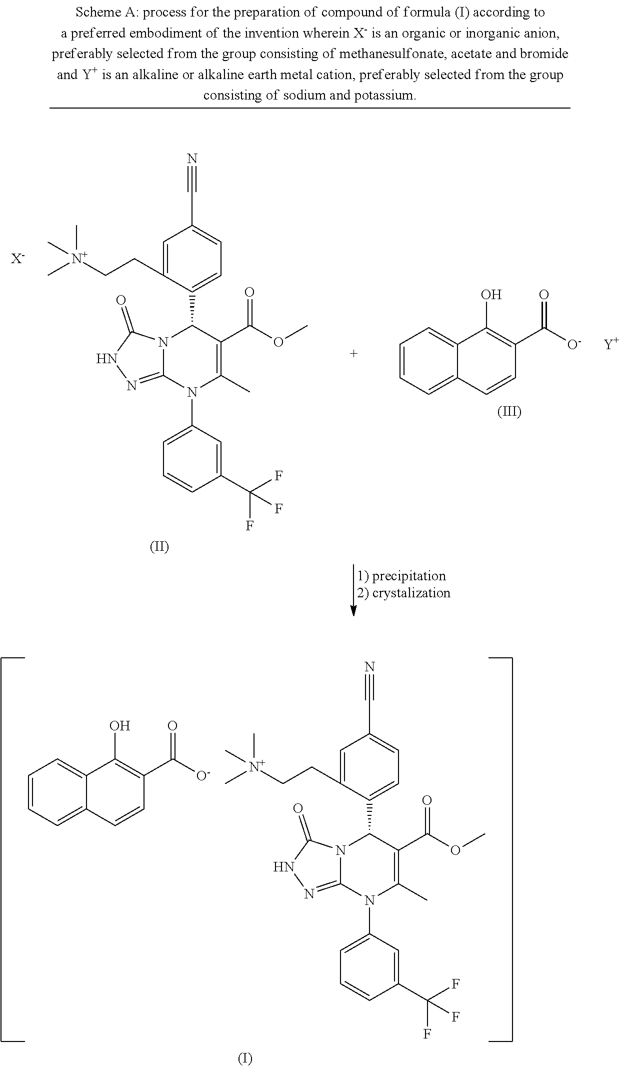

The compound of the invention of formula (I) may be prepared from (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-methanesulfonate of formula (II) as reported in Example 1, obtained according e.g. to the procedures described in WO 2014/095700, by dissolution in an aqueous or organic solvent, such as water, and addition with a solution of sodium or potassium xinafoate of formula (III) in an appropriate aqueous or organic solvent, preferably water, under mechanical stirring, thus obtaining the precipitation of the xinafoate salt, which is filtered and washed with an aqueous or organic solvent, such as water or acetone, then adding an aqueous or organic solvent, preferably acetone, preferably in the interval of 2-10 ml, and sonicating for a suitable period of time, preferably in the interval of 5-20 min, filtered and dried under vacuum, to obtain a crystal form.

Similarly the salt of the compound of the invention of formula (I) could be obtained using any water soluble salt of (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium.

Processes which can be used and are described and reported in Examples should not be viewed as limiting the scope of the synthetic methods available for the preparation of the compound of the invention.

Compounds used as starting materials or intermediates may be commercially available, their preparation may be specifically described in the literature or they may be prepared according to methods available in the literature and well known to the person skilled in the art.

The process described is particularly advantageous as it is susceptible of being properly modulated for reaching the desidered product synthetic purity, through any proper variant known to the skilled person, so as to obtain the desired compounds of the invention. Such variants are comprised within the scope of the present invention.

The following examples illustrate the invention without limiting its scope.

### EXAMPLES

### Abbreviations Used in the Experimental Section

RT Room temperature
DMSO Dimethylsulphoxide
PBS phosphate buffered saline
BAL bronchoalveolar lavage
eDacq USB Data Acquisition
XRPD X-Ray Powder Diffraction
NMR Nuclear Magnetic Resonance Spectroscopy

### X-Ray Powder Diffraction (XRPD)

The crystalline state of sample was investigated by X-ray powder diffraction (Empyrean V2.0, Panalytical) equipped with Cu radiation source (Cu Kα λ=1.5406 Å). Samples were placed on Si zero background sample holders spinning with revolution time 4s. The measurements were performed in reflection mode, 2Theta scan from 1.5 to 45°, step size 0.02°, soller slit 0.02 rad, divergence slit ⅛°, antiscatter slit ¼°.

### Nuclear Magnetic Resonance Spectroscopy (1H NMR)

All the 1H NMR spectra were performed on a Bruker AVANCE III HD 600 spectrometer operating at 600 MHz (proton frequency). The spectrometer was equipped with a 5 mm TCI INVERSE TRIPLE RESONANCE CRYOPROBE H-C/N-D-0.5-Z ATMA. The probe is fitted with an actively shielded single axis Z-gradient and allowed simultaneous decoupling on multiple X-nuclei such as 13C and 15N as well as automatic tuning and matching.

### Example 1

### (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium xinafoate

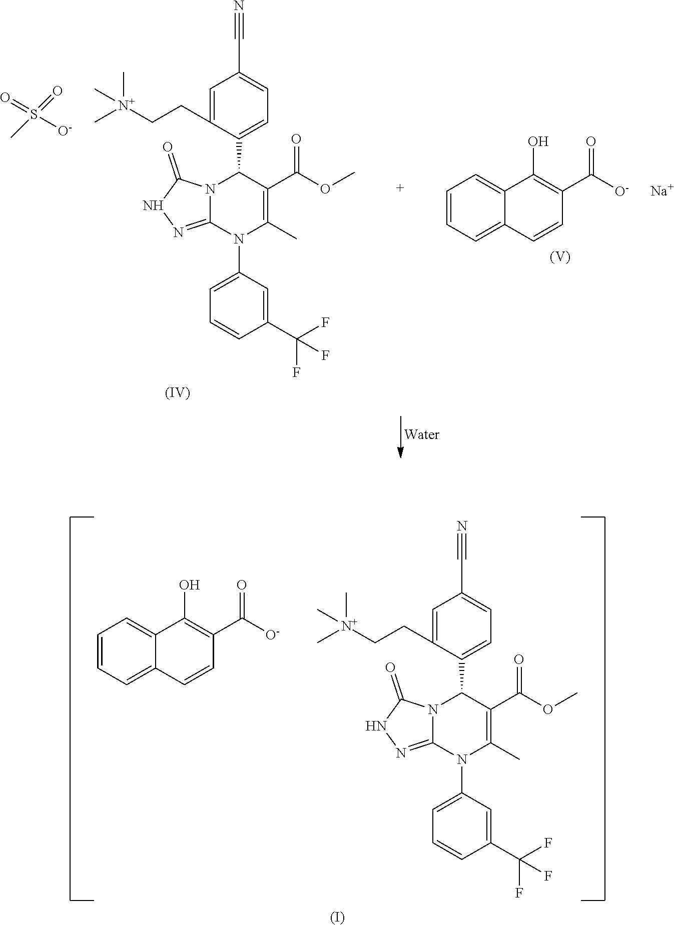

(2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium methanesulfonate (IV) (50 mg; 0.08 mmol), prepared as described in WO 2014/095700, was dissolved in water (2 ml) and added with a solution of sodium xinafoate (V) (15 mg, 0.08 mmol) in water (1 ml) under mechanical stirring, obtaining the precipitation of the xinafoate salt as an amorphous solid. The amorphous solid was been washed twice with 3 ml of water, then adding 5 ml of Acetone and sonicating for 10 minutes. A white solid precipitated. The solid was filtered and dried under vacuum at 25° C. affording 55 mg of a solid (95% yield). Stoichiometric ratio between (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium cation and xinafoate anion is 1:1 and it is confirmed by NMR and XRPD.

¹H NMR (400 MHz, DMSO-d₆) δ ppm 2.16 (s, 3H), 3.21 (s, 9H), 3.35-3.44 (m, 1H), 3.51 (s, 3H), 3.64-3.79 (m, 2H), 3.93-4.03 (m, 1H) 6.22 (s, 1H) 6.91 (d, J=8.38 Hz, 1H), 7.28 (ddd, 1H), 7.37 (ddd, 1H) 7.64-7.78 (m, 4H), 7.79-7.86 (m, 2H), 7.88-7.98 (m, 2H), 8.09 (s, 1H), 8.14 (d, 1H) 11.26 (s, 1H)

XRPD: 8.6, 9.9, 10.7, 11.0, 13.0, 15.3, 19.3, 19.7, 23.2, 27.6±0.2 degrees/2 theta

### Solubility in water of (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium methanesulfonate (IV)

200 mg of (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium methanesulfonate (IV) were placed in 1 ml of water at RT. The complete dissolution of the material was visually observed.

The visual observation of an instantaneous dissolution was index that the methanesulfonate of formula (IV) has a solubility >200 mg/ml.

### Solubility in water of (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium xinafoate (I)

2 mg of (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium xinafoate (I) were placed in 1 ml of water at RT and diluited with additional water until dissolution. From this experiment the xinafoate salt has a solubility lower than 0.05 mg/ml.

The results of the solubility tests are summarized in the following Table 1.

TABLE 1
Solubility in water of different salts

| Salt | Solubility (mg/ml) |
|---|---|
| Methanesulfonate | >200 |
| Xinafoate | <0.05 |

The above Table 1 clearly shows a significantly higher solubility of the methanesulfonate salt with respect to the xinafoate salt.

### Biological Assay

The efficacy of different salts of (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium was evaluated in the in vivo model of lung injury induced by human neutrophil elastase (HNE).

In addition, the effects of different (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium salts on lung function parameters were also assessed in rats by head-out plethysmography in order to compare the potential local adverse effects of each salt of (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium.

### Example 2.1/Comparative

### HNE-Induced Lung Injury Assay

Male Sprague Dawley rats were dosed by nose-only inhalation with either vehicle (lactose), (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium methanesulfonate salt or (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium xinafoate salt given as dry powder 3 hours prior to phosphate buffered saline (PBS) (control group, PBS alone treated animals) or HNE intratracheal (i.t.) administration (100 U/rat). Then, one hour after the PBS or HNE administration animals were sacrificed and bronchoalveolar lavage (BAL) was performed in order to assess the HNE-induced lung injury measured as haemoglobin concentration.

BAL fluid samples were centrifuged at 800 g for 15 min at 4° C. The supernatant was collected and the pellet was re-suspended in 3 mL of distilled water. A standard curve with known volumes of lysated blood cells was made from a stem solution of lysated blood cells. 150 μL of standards and samples were transferred in duplicate to a 96-well plate and the OD measured at 412 nm.

Percentage of compound efficacy (assessed as inhibition of HNE-induced haemoglobin content in BALF) was calculated following this formula: 100−[(mean of haemoglobin concentration of test compound treated rats exposed to HNE)−(mean of haemoglobin concentration of vehicle treated rats exposed to PBS)]/[(mean of haemoglobin concentration of vehicle treated rats exposed to HNE)−(mean of haemoglobin concentration of vehicle treated rats exposed to PBS)]×100.

### Results

In this model, HNE i.t. challenge induces a significant increase of BAL fluid haemoglobin content when compared to control group (0 g/dL for control group and 0.19 g/dL for HNE group, p<0.001). (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium methanesulfonate and xinafoate salts administered by inhalation as dry powder formulation at three different doses (0.03, 0.3 and 0.6 mg/kg for methanesulfonate and xinafoate salts) showed a dose-dependent inhibition of BAL fluid haemoglobin content. Specifically, the methanesulfonate salt showed an inhibition ranging from 20% at 0.3 mg/kg and 25% at 0.6 mg/kg when compared to the HNE-treated vehicle control. Similarly, the xinafoate salt induced a BAL fluid haemoglobin content reduction ranging from 35% at 0.3 mg/kg (p<0.05) and 80% at 0.6 mg/kg (p<0.001) in comparison with the HNE-treated vehicle control.

These data demonstrate that the two salts of (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium are able to inhibit HNE in vivo, and surprisingly the xinafoate salt shows a superior efficacy than the methanesulfonate salt in this model.

The results of the HNE-induced lung injury assay are summarized in the following Table 2.

TABLE 2
Percentage of inhibition of different salts in the HNE-
induced lung injury assay calculated as defined above

| | % Inhibition at 300 | % Inhibition at 600 |
|---|---|---|
| Salt | μg/Kg | μg/Kg |
| Methanesulfonate | 20 | 25 |
| Xinafoate | 35 | 80 |

The above Table 2 shows that both the xinafoate and methanesulfonate salts are able to inhibit HNE in vivo. Further despite the lower solubility, the xinafoate salt shows a superior activity in the above described pharmacodynamic model, with respect to the methanesulfonate salt.

### Example 2.2/Comparative

### Head-Out Plethysmography (HOP) Assay

Male Wistar rats were dosed by the snout-only inhalation route with (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium methanesulfonate salt (described in WO 2014/095700), (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium xinafoate salt (I) and (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium acetate salt (which is described in WO 2014/095700), (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium bromide salt (whose synthesis is described in WO 2014/095700) or lactose (vehicle) given as dry powder formulations. The duration of aerosol exposure was of 60 minutes. On the day of dosing, animals were placed into the plethysmograph tubes for at least 30 minutes prior to dosing and respiratory parameters: respiratory rate, tidal volume, and PenH were continuously recorded for at least 30 minutes pre-dose, 60 minutes during dosing (exposure) and 90 minutes post-exposure. The respiratory parameters were recorded every minute for a total period of 3 hours using the EMMS eDacq system (PLY231, EMMS, Bordon, United Kingdom). The effect of the test compounds on different lung function parameters was measured as % of change versus vehicle (lactose) group and was reported at peak effect (i.e. the highest effect observed).

### Results

A single inhaled administration of two doses of (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium methanesulfonate salt (0.6 and 6 mg/kg) as dry powder produced statistically significant changes in all three respiratory parameters analysed and these effects were mainly observed during exposure. Specifically, a significant increase of respiratory rate was observed compared to vehicle (lactose) treated rats at both doses during inhalation with a peak effect of 32% of increase observed at the dose of 0.6 mg/kg and a maximum increase of 54% induced by the dose of 6 mg/kg. The increase of respiratory rate observed during inhalation of (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium methanesulfonate salt was associated with a significant reduction of tidal volume of 35% and 39% (peak effect at the doses of 0.6 and 6 mg/kg, respectively) and a significant and dose-dependent increase of PenH of about 8-fold for the low dose and 16-fold for the high dose compared to vehicle group.

Similar effects were observed with (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium bromide salt (0.55 and 5.5 mg/kg), which produced statistically significant reduction in tidal volume during inhalationcompared to vehicle, with a maximum reduction of 38% for the high dose group. This decrease in tidal volume also coincided with a statistically significant increase in PenH area of about 8-fold for the low dose and 20-fold for the high dose compared to vehicle, and a significant increase in respiratory rate again during inhalation with the low dose reaching a maximum effect of 59% compared to vehicle.

Likewise, (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium acetate salt administered via single snout-only inhalation at 7 mg/kg produced statistically significant reduced tidal volume when compared to the vehicle control with a maximum decrease of 50%. This salt also induced a statistically significant increase in respiratory rate reaching a maximum effect of 51%. These changes were associated with a significant increase in PenH area of about 9-fold.

The peak effects for all three (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium salts were observed after 20-50 minutes of compound inhalation with return to baseline values occurring immediately after dosing.

Conversely, (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium xinafoate salt administered via single snout-only inhalation at 8 and 25 mg/kg as dry powder did not alter any lung function parameters either during or after dosing.

The results of the Head-out plethysmography (HOP) assay are summarized in the following Table 3.

TABLE 3
Percentage of reduction of tidal volume at peak effect induced by
the different salts in the Head-out plethysmography (HOP) assay

| | % of Reduction of tidal volume |
|---|---|
| Salt | at the highest tested dose |
| Methanesulfonate | 35-39 |
| Bromide | 38 |
| Acetate | 50 |
| Xinafoate | No variation of any lung function parameters |

These data demonstrate that conversely to the other salts, the xinafoate salt does not affect any lung function parameters, suggesting that this salt is particularly suitable for administration, also in terms of patients tolerability and local adverse effects profile.

1. The compound (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4,3-a]pyrimidin-5-yl]-phenyl}-ethyl)-trimethyl-ammonium xinafoate of formula (I)

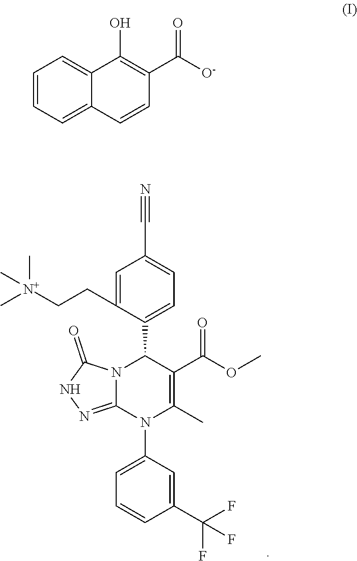

2. A process for the preparation of the compound of formula (I) according to claim 1, comprising:
(1) dissolving in water (2-{5-Cyano-2-[(R)-6-methoxycarbonyl-7-methyl-3-oxo-8-(3-trifluoromethyl-phenyl)-2,3,5,8-tetrahydro-[1,2,4]triazolo[4.3-a]pyrimidin-5-yl]-phenyl}-ethyl)-trimethyl-ammonium salt (II) 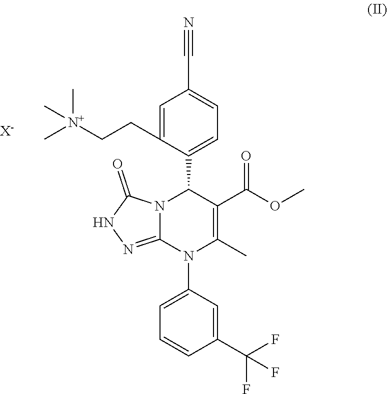
wherein X⁻ is an organic or inorganic anion, to obtain a first solution and
(2) mixing said first solution with a second solution of a xinafoate salt of formula (III) 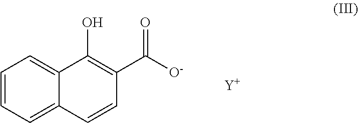
wherein Y⁺ is an alkaline or alkaline earth metal cation, to obtain the product.

3. A process according to claim 2, which further comprises:
(3) washing the product with one or more aqueous or organic solvents.

4. A process according claim 2, wherein X⁻ is bromide, methanesulfonate or acetate.

5. A process according to claim 2, wherein Y⁺ is sodium or potassium.

6. A crystal form of a compound of formula (I) according to claim 1, wherein said crystal form is characterized by at least one of the following XRPD peaks: 8.6, 9.9 and 23.2±0.2 degrees/2 theta [Cu Kα radiation (λ=1.5406 Å)].

7. A crystal form according to claim 6, characterized by the following XRPD peaks: 8.6, 9.9, 10.7, 11.0 and 23.2±0.2 degrees/2 theta [Cu Kα radiation (λ=1.5406 Å)].

8. A crystal form of a compound of formula (I) according to claim 6, characterized by the following XRPD peaks: 8.6, 9.9, 10.7, 11.0, 13.0, 15.3, 19.3, 19.7, 23.2 and 27.6±0.2 degrees/2 theta [Cu Kα radiation (λ=1.5406 Å)].

9. A crystal form of a compound of formula (I) obtained according to a process according to claim 3.

10. A pharmaceutical composition comprising the compound of formula (I) according to claim 1 and one or more pharmaceutically acceptable carriers and/or excipients.

11. A pharmaceutical composition comprising the crystal form of a compound of formula (I) according to claim 6 and one or more pharmaceutically acceptable carriers and/or excipients.

12. A pharmaceutical composition according to claim 10, formulated in form of a dry powder.

13. (canceled)

14. A method for the prevention and/or treatment of an inflammatory or obstructive respiratory disease, comprising administering to a subject in need thereof an effective amount of the compound of formula (I) according to claim 1.

15. The method according to claim 14, wherein the inflammatory or obstructive respiratory disease is selected from asthma, chronic obstructive pulmonary disease (COPD), bronchiectasis, chronic bronchitis, lung fibrosis, idiopathic pulmonary fibrosis, pneumonia, acute respiratory distress syndrome (ARDS), pulmonary emphysema, smoking-induced emphysema and cystic fibrosis.

16. A method for the prevention and/or treatment of an inflammatory or obstructive respiratory disease, comprising administering to a subject in need thereof an effective amount of a pharmaceutical composition according to claim 10.

17. The method according to claim 16 wherein the inflammatory or obstructive respiratory disease is selected from: asthma, chronic obstructive pulmonary disease (COPD), bronchiectasis, chronic bronchitis, lung fibrosis, idiopathic pulmonary fibrosis, pneumonia, acute respiratory distress syndrome (ARDS), pulmonary emphysema, smoking-induced emphysema and cystic fibrosis.

18. A process according claim 3, wherein X⁻ is bromide, methanesulfonate or acetate.

19. A process according to claim 3, wherein Y⁺ is sodium or potassium.
